(19) United States
(12) Patent Application Publication
(10) Pub. No.: US 2014/0058330 A1
(43) Pub. Date: Feb. 27, 2014

(54) Dental implant device providing intravascular access

(71) Applicant: Irwin N. Boe, Leawood, KS (US)

(72) Inventor: Irwin N. Boe, Leawood, KS (US)

(21) Appl. No.: 13/573,139

(22) Filed: Aug. 25, 2012

(51) Int. Cl. A61M 25/04

(52) U.S. Cl. 604/174

(57) ABSTRACT

A dental implant device (30) providing intravascular access to a body of a patient. The device (30) includes a portal component (32) installed in a mouth (50) of the patient in place of or in association with a natural tooth, and a removable cap (34) resembling a natural tooth. The portal component (32) is connectable to an implanted catheter (36) which may extend from the device (30) into the body via vasculature associated with the periodontal ligament.

### BACKGROUND OF THE INVENTION

1. Technical Field

The present invention relates to intravascular catheters. More specifically, the present invention concerns a dental implant device providing intravascular access to a body.

2. Background

Intravascular catheters are employed in modern-day medical practice to provide access to patients' vascular systems, particularly for the purpose of introducing or withdrawing fluids. As such, health-care institutions employ millions of intravascular catheters each year. However, the use of such catheters puts patients at risk for local and systemic infections, including local site infection, catheter-related bloodstream infection (CRBSI), septic thrombophlebitis, endocarditis, and other metastatic infections (e.g., lung abscesses, brain abscesses, osteomyelitis, and endophthalmitis), and other complications.

The incidence of CRBSI varies considerably by type of catheter, frequency of catheter manipulation, and patient-related factors (e.g., underlying disease and acuity of illness). Peripheral venous catheters are the devices most frequently used for vascular access. Although the incidence of local or bloodstream infections (BSIs) associated with peripheral venous catheters is usually low, serious infectious complications produce considerable annual morbidity because of the frequency with which such catheters are used. However, the majority of serious catheter-related infections are associated with central venous catheters (CVCs), especially those that are placed in patients in intensive care units (ICUs).

In the ICU setting, the incidence of infection is often higher than in the less acute in-patient or ambulatory setting. In the ICU, central venous access might be needed for extended periods of time, patients can be colonized with hospital-acquired organisms, and the catheter can be manipulated multiple times per day for the administration of fluids, drugs, and blood products. Moreover, when catheters are inserted in urgent situations, optimal attention to aseptic technique might not be feasible. Certain catheters (e.g., pulmonary artery catheters and peripheral arterial catheters) can be accessed multiple times per day for hemodynamic measurements or to obtain samples for laboratory analysis, augmenting the potential for contamination and subsequent clinical infection.

Therefore, by several analyses, the cost of CVC-associated BSI is substantial, both in terms of morbidity and in terms of financial resources expended. To improve patient outcome and reduce health-care costs, strategies should be implemented to reduce the incidence of these infections. Several strategies have been studied and shown to be effective in reducing CRBSI, studies using multiple strategies have not been conducted.

A total of 250,000 cases of CVC-associated BSIs have been estimated to occur annually if entire hospitals are assessed rather than ICUs exclusively. In this case, attributable mortality is an estimated 12% to 25% for each infection, and the marginal cost to the health-care system is $25,000 per episode.

The magnitude of the potential for CVCs to cause morbidity and mortality resulting from infectious complications has been estimated in several studies. In the United States, 15 million CVC days (i.e., the total number of days of exposure to CVCs by all patients in the selected population during the selected time period) occur in ICUs each year. If the average rate of CVC-associated BSIs is 5.3 per 1,000 catheter days in the ICU, then approximately 80,000 CVC-associated BSIs occur in ICUs each year in the United States. The attributable mortality for these BSIs has ranged from no increase in mortality in studies that controlled for severity of illness, to 35% increase in mortality in prospective studies that did not use this control. Thus, the attributable mortality remains unclear. The attributable cost per infection is an estimated $34,508 to $56,000, and the annual cost of caring for patients with CVC-associated BSIs ranges from $296 million to $2.3 billion.

Due to these and other problems and disadvantages in the prior art, a need exists for improvements in catheter design.

### SUMMARY OF THE INVENTION

The present invention overcomes the above-identified and other problems and disadvantages by providing a dental implant device providing intravascular access to the body of a patient. The device may be used, for example, for introducing antibiotics, pain medicine, chemotherapy, or nutrition or for withdrawing blood samples with less risk of infections and other complications than prior art catheters.

Broadly, the dental implant access device comprises an portal component installable in a mouth of the patient, with the portal component having a first end and a second end, and with the second end being connected to a tube implanted in the body of the patent; and a cap removably attachable over the first end of the portal component, wherein, when the cap is removed, fluid, gas, or other matter can be introduced into the portal component for distribution within the body of the patient via the implanted tube.

In various embodiments, the device may further comprise one or more of the following features. The portal component may be installable in the mouth of the patient in place of a natural tooth or in association with an existing natural tooth. The first end of the portal component may include a septum for receiving a needle containing the matter, or it may include a connection which is connectable to a reservoir of the matter. The portal component may include an internal reservoir for receiving and releasing over time an amount of the matter. The cap may be shaped, colored, or otherwise constructed to resemble at least a crown portion of a natural tooth. The tube may be a tunneled catheter. The portal component may be installable adjacent to a vasculature associated with a periodontal ligament.

These and other features of the present invention are discussed in greater detail in the section below entitled DETAILED DESCRIPTION OF THE INVENTION.

### BRIEF DESCRIPTION OF THE DRAWINGS

The present invention is described herein with reference to the following drawing figures, which are not necessarily to scale:

FIG. 1 is across-sectional elevation view of a natural tooth and its surrounding tissues and structures;

FIG. 2 is an exploded cross-sectional elevation view of the device of the present invention installed in a patient's mouth; and

FIG. 3 is a plan view of the portal component of the device of FIG. 2 installed in the patient's mouth in place of an existing tooth.

### DETAILED DESCRIPTION OF THE INVENTION

With reference to the figures, a dental implant device 30 is herein described, shown, and otherwise disclosed in accordance with one or more embodiments of the present invention, including one or more preferred embodiments.

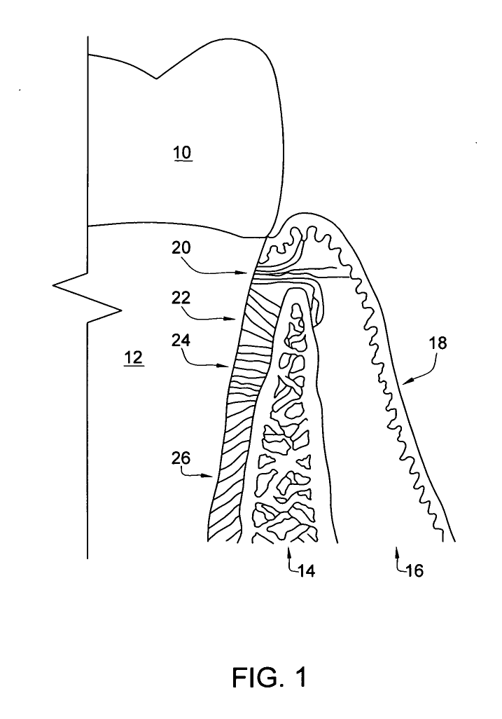

FIG. 1 illustrates an example natural tooth and its surrounding tissues and structures, including a clinical crown 10, a tooth root (cementum) 12, an alveolar bone 14, a subepithelial connective tissue 16, an oral epithelium 18, principle gingival fibers 20, and alveolar crest fibers 22, horizontal fibers 24, and oblique fibers 26 of the periodontal ligament. The periodontal ligament is a system of collagenous connective tissue fibers that attaches the root of a tooth to its alveolus of bone by way of Sharpey's fibers. It contains blood vessels, lymph vessels, and nerves, and consists of five groups of fibers, including interdental, alveolar crestal 22, horizontal 24, oblique 26, and apical and possibly interradicular fibers if the tooth is multirooted.

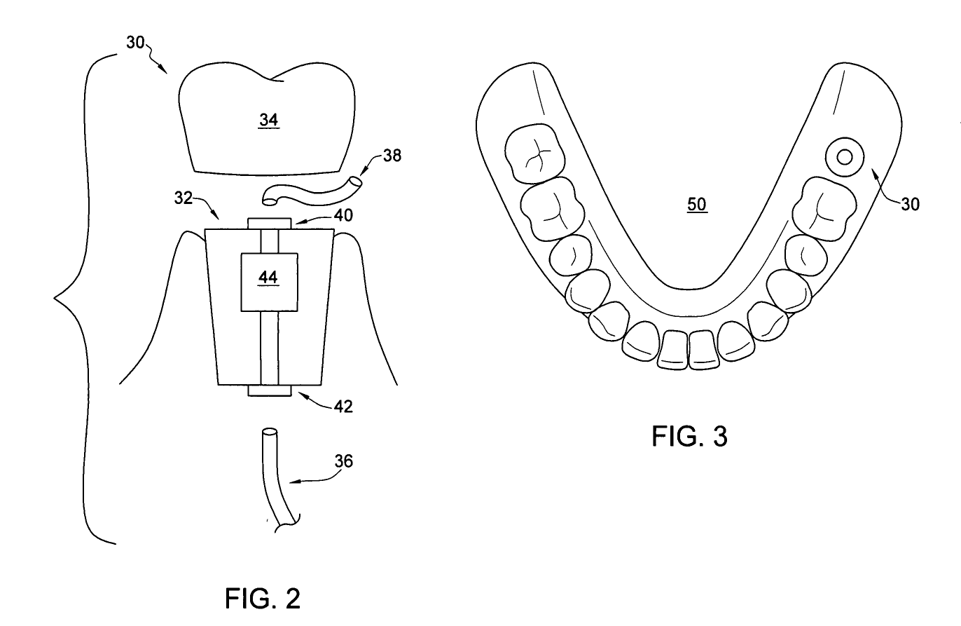

Referring to FIG. 2, in one embodiment the device 30 broadly comprises a portal component 32 and a cap 34. As seen in FIG. 3, the portal component 32 is installed in a patient's mouth 50 in association with or in place of a tooth and provides an portal between an end of a tunneled or otherwise implanted tube of a catheter 36 and an external delivery system 38. The cap 34 provides for protectively covering or selectively exposing a portion or all of the portal component 32. In use, the portal component 32 is installed within the patient's mouth 50, and the cap 34 is placed protectively over the portal component 32. The catheter 36 is then implanted and connected to the portal component 32. As needed, the cap 34 is removed and the portal component 32 is connected to the delivery system 38 in order to deliver matter such as antibiotics, pain medicine, chemotherapy, or nutrition or for withdrawing blood samples via the implanted tube 36, with less risk of infections and other complications than prior art catheters.

In various embodiments, the device 30 may include any one or more of the following features. The portal component 32 may be installable in the mouth of the patient either in place of a natural tooth or in association with an existing natural tooth, and may be constructed of any suitable material, such as plastic, ceramic, metal, or a combination thereof. The portal component 32 may have a first end 40 and a second end 42. The first end 40 may be associated with the delivery system 38, and may include a septum for receiving a needle for injecting the matter or a connection which is connectable to an external reservoir of the matter. The second end 42 may be connectable to the implanted catheter tube 36. The portal component 32 may include an internal reservoir 44 for receiving and releasing over time an amount of the matter.

The cap 34 may broadly comprise a protective covering which is selectively removable from the portal component 32; constructed of any suitable material, such as plastic, ceramic, metal, or a combination thereof; and having any suitable shape, such as a shape resembling at least a portion (e.g., a crown portion) of a tooth or type of tooth normally located where the device 30 is installed in the patient's mouth. The cap 34 substantially prevents any foreign material from entering or otherwise corrupting the portal component 32. When the cap 34 is shaped to resemble a tooth, the entire device 30 can go substantially unnoticed by the patient or others when not in use. As such, the cap 34 may be similar to a conventional dental crown.

The catheter 36 may be a tunneled catheter. The catheter 36 may extend from the device 30 into the body of the patient via vasculature associated with the periodontal ligament and into, for example, the jugular vein, subclavian vein, or superior vena cava. For many applications, the catheter will terminate in the superior vena cava, just upstream of the right atrium. This position allows infused agents to spread throughout the body quickly and efficiently.

Although the mouth is generally thought of as being awash in bacteria, it is also continuously self-cleaning. Furthermore, by accessing the vascular system via the periodontal ligament, the dental implant device's tunneling components remain subcutaneous, thereby minimizing infections and other complications that can arise with conventional intravascular catheters that compromise the skin.

Although the invention has been disclosed with reference to various particular embodiments, it is understood that equivalents may be employed and substitutions made herein without departing from the scope of the invention as recited in the claims.

Having thus described the preferred embodiment of the invention, what is claimed as new and desired to be protected by Letters Patent includes the following:

1. A dental implant device providing intravascular access to a body of a patient, the dental implant device comprising:
a portal component installed in a mouth of the patient in place of a natural tooth, with the portal component having a first end and a second end; and with the second end being connected to a catheter implanted in the body of the patent; and
a cap constructed to resemble a crown portion of the natural tooth, and removably attachable over the first end of the portal component,
wherein, when the cap is removed, matter can be introduced into the portal component for distribution within the body of the patient via the catheter.

2. A dental implant device providing intravascular access, the dental implant device comprising:
a portal component installable in a mouth of a patient, with the portal component having a first end and a second end, and with the second end being connected to a tube implanted in a body of the patent; and
a cap constructed to resemble at least a portion of a natural tooth, and removably attachable over the first end of the portal component,
wherein, when the cap is removed, matter can be introduced into the first end of the portal component for distribution within a body of the patient via the tube.

3. The dental implant device as set forth in claim 2, wherein the portal component is installable in the mouth of the patient in place of a natural tooth.

4. The dental implant device as set forth in claim 2, wherein the portal component is installable in the mouth of the patient in association with an existing tooth.

5. The dental implant device as set forth in claim 2, wherein the first end includes a septum for receiving a needle containing the matter.

6. The dental implant device as set forth in claim 2, wherein the first end includes a connection which is connectable to a reservoir of the matter.

7. The dental implant device as set forth in claim 2, wherein the portal component includes an internal reservoir for receiving and releasing over time an amount of the matter.

8. The dental implant device as set forth in claim 2, wherein the cap is shaped and colored to resemble at least a crown portion of a natural tooth.

9. The dental implant device as set forth in claim 2, wherein the tube is a tunneled catheter.

10. The dental implant device as set forth in claim 2, wherein the portal component is installable adjacent to a vasculature associated with a periodontal ligament.

11. A dental implant device providing intravascular access to a body of a patient, the dental implant device comprising:
a portal component installed in a mouth of the patient in place of a natural tooth and adjacent to a vasculature associated with a periodontal ligament, with the portal component having a first end and a second end, and with the second end being connected to a catheter which runs through the vasculature; and
a cap constructed to resemble a crown portion of the natural tooth, and removably attachable over the first end of the portal component,
wherein, when the cap is removed, matter can be introduced into the portal component for distribution within the body of the patient via the catheter.
